(19) United States
(12) Patent Application Publication
(10) Pub. No.: US 2026/0165645 A1
(43) Pub. Date: Jun. 18, 2026

(54) Continuous Vital Sign Monitoring and Automatic Opioid Overdose Reversal Wearable Device

(71) Applicant: Nathaniel Tong, Dahlonega, GA (US)

(72) Inventor: Nathaniel Tong, Dahlonega, GA (US)

(21) Appl. No.: 19/095,102

(22) Filed: Mar. 31, 2025

Related U.S. Application Data
(60) Provisional application No. 63/733,139, filed on Dec. 12, 2024.

(51) Int. Cl. A61B 5/00; A61B 5/0205; A61B 5/021; A61B 5/024; A61B 5/0245; A61B 5/08; A61B 5/11; A61B 5/1455; A61M 37/00

(52) U.S. Cl. CPC A61B 5/4836; A61B 5/0205; A61B 5/02141; A61B 5/02416; A61B 5/02438; A61B 5/0245; A61B 5/0803; A61B 5/1102; A61B 5/14551; A61B 5/681; A61B 5/6831; A61B 5/7405; A61B 5/742; A61B 5/746; A61B 5/7475; A61M 37/0015; A61B 2560/0468; A61B 2562/0247; A61B 2562/0261; A61M 2037/0023; A61M 2202/0007; A61M 2202/0468; A61M 2205/0294; A61M 2205/054; A61M 2205/10; A61M 2205/18; A61M 2205/3306; A61M 2205/3327; A61M 2205/3344; A61M 2205/3592; A61M 2205/502; A61M 2205/505; A61M 2205/581; A61M 2209/088; A61M 2230/005; A61M 2230/04; A61M 2230/20; A61M 2230/40

(57) ABSTRACT

The present invention relates to a wearable health monitoring device designed to detect and respond to life-threatening emergencies, including opioid overdoses and respiratory failure. The device comprises a plurality of sensors to continuously monitor oxygen levels, heart rate, blood pressure, and respiration patterns. A microcontroller processes real-time sensor data to detect abnormalities and trigger alerts. If the user remains unresponsive, an automatic intervention system activates, which includes a mild electric shock mechanism to stimulate the nervous system and a built-in naloxone injector to administer an opioid antidote. Additionally, a wireless communication module transmits emergency notifications and GPS coordinates to emergency responders and pre-set contacts. The device prevents overdose-related deaths and improves emergency medical care.

### CROSS-REFERENCE TO RELATED APPLICATION

The present application claims priority to, and the benefit of, U.S. Provisional Application No. 63/733,139, which was filed on Dec. 12, 2024, and is incorporated herein by reference in its entirety.

### FIELD OF THE INVENTION

The present invention generally relates to wearable health monitoring devices and emergency medical response systems. More specifically, the invention relates to a smart wristband that continuously monitors vital signs, detects life-threatening emergencies, and autonomously administers life-saving interventions when necessary. The wearable device is designed to be worn on the wrist, ankle, or other body parts, enabling continuous tracking of oxygen levels, heart rate, blood pressure, and respiration patterns. In some embodiments, the device includes a built-in automatic naloxone injector that administers a life-saving dose in response to an opioid overdose, as well as an electric stimulation mechanism to assist in restoring breathing. Accordingly, this disclosure makes specific reference thereto the present invention. Nonetheless, it is to be appreciated that aspects of the present invention are also equally applicable to other like applications, devices, and methods of manufacture.

### BACKGROUND

By way of background, many individuals are addicted to opioids which has become a severe public health crisis and leads to overdose-related fatalities. Individuals struggling with opioid dependency are at high risk of respiratory failure, often occurring when they are alone or in environments where immediate medical assistance is unavailable. When an individual overdoses, their breathing may slow down or stop completely, a condition known as opioid-induced respiratory depression (OIRD). Without timely intervention, the lack of oxygen can cause irreversible brain damage or death within minutes.

Beyond opioid overdoses, other conditions such as untreated sleep apnea or cardiac-related respiratory failure can also result in sudden cessation of breathing, requiring immediate emergency response. Traditional medical interventions rely on external assistance, such as emergency responders administering drugs or performing CPR, which may not be accessible in time. Accordingly, there is a critical need for an automated, real-time monitoring and intervention system capable of detecting respiratory distress, alerting emergency services, and administering life-saving treatments autonomously.

Therefore, there exists a long-felt need in the art for a wearable health monitoring device that can autonomously detect opioid overdoses, respiratory failure, and other life-threatening emergencies in real-time. There is a long-felt need for a device that continuously tracks vital signs, such as oxygen levels, heart rate, blood pressure, and respiration patterns, and automatically alerts emergency services when an individual is unresponsive. Additionally, there is a long-felt need for a wearable intervention system that not only detects emergencies but also administers life-saving treatment. Furthermore, there is a long-felt need for a device that integrates wireless connectivity to transmit real-time health data and GPS location to emergency responders. More specifically, there exists a long-felt need for a smart wristband that provides both automated and user-confirmed emergency response mechanisms. Also, there is a long-felt need for an energy-efficient, portable, and comfortable device that can be worn daily without causing discomfort. Finally, there is a long-felt need for a comprehensive health monitoring and emergency intervention solution that can be used by opioid users, sleep apnea patients, and individuals at risk of respiratory distress.

The subject matter disclosed and claimed herein, in one embodiment, comprises a wearable health monitoring device that is designed to track vital signs, detect emergencies, and automatically administer life-saving interventions when needed. The device is configured as a wrist-worn smart band and includes a touchscreen display that shows real-time health data, emergency alerts, and interactive prompts. The device incorporates a plurality of sensors, including a pulse oximeter, blood pressure sensor, heart rate sensor, and respiration sensor, that continuously monitor the wearer's health. Upon detecting a critical drop in oxygen levels or cessation of breathing, the device issues an audible and vibration alert to prompt user interaction. If the wearer does not respond, the device activates a built-in naloxone injector for opioid overdose treatment and an electric stimulation module to restore breathing.

In this manner, the wearable health monitoring device of the present invention fulfills the long-felt need for an autonomous life-saving system that can detect and respond to opioid overdoses and respiratory distress in real-time. The device integrates continuous health monitoring, emergency intervention, and wireless connectivity, thereby providing medical help as quickly as possible, even if the wearer is alone. The automated naloxone injection system and mild electric stimulation module offer immediate first-line treatment, significantly reducing the risk of fatal overdose. The device can be worn comfortably and securely, making it ideal for daily use by individuals. The device helps save lives for anyone that may stop breathing including, but not limited to, opioid addicts, undiagnosed sleep apnea patients, and more.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wearable health monitoring device. The device comprises a housing configured to be worn on a body part of a user, a touchscreen display disposed on the housing, the touchscreen display is configured to display vital signs, emergency alerts, and interactive prompts, a plurality of sensors are configured to continuously monitor at least one vital sign of the user, the plurality of sensors comprises at least a pulse oximeter sensor for measuring oxygen saturation levels in the blood, a blood pressure sensor for measuring the user's blood pressure, a heart rate sensor for monitoring the user's heart rate and rhythm, and a respiration sensor for detecting breathing patterns of the user.

In another aspect, a wearable health monitoring device is disclosed. The device includes a sensor system configured to monitor the vital signs of a user and detect opioid overdose based on oxygen saturation, heart rate, and respiration patterns. An automatic intervention module comprises an electric shock mechanism configured to deliver a mild electric shock to stimulate the user's nervous system when respiratory failure is detected and a miniature naloxone injector to deliver a dose of naloxone subcutaneously or intramuscularly.

In one embodiment, the device includes a speaker to provide an audible alert when an abnormal vital sign is detected and a wireless communication module is configured to transmit emergency notifications and GPS location data to emergency services when the user is unresponsive.

In yet another aspect, the miniature naloxone injector comprises a retractable micro-needle injector configured to deliver naloxone subcutaneously or intramuscularly, a miniature motor configured to activate the micro-needle injector upon detection of an opioid overdose, and a drug reservoir configured to store at least one dose of naloxone.

In still another embodiment, a method for detecting and responding to opioid overdoses using a wearable health monitoring device is described. The method includes the steps of continuously monitoring a user's oxygen saturation, heart rate, and respiration patterns using a plurality of sensors, detecting a critical drop in oxygen levels and an absence of normal respiratory activity for a predefined duration, generating an alert signal that requires the user to confirm consciousness via a touchscreen prompt or physical interaction with the device, determining that the user is unresponsive if no response is received within a predetermined time period, automatically activating a built-in injection system comprising a retractable micro-needle injector and a miniature motor, wherein the micro-needle injector delivers a dose of naloxone to counteract the effects of an opioid overdose, and transmitting an emergency notification to emergency contacts and medical personnel, including real-time GPS location and vital sign data.

In another embodiment, the miniature naloxone injector is configured to determine the appropriate amount of naloxone to administer based on the wearer's physiological parameters and can administer a second dose if the first dose does not restore normal breathing.

In yet another embodiment, the wearable device distinguishes opioid overdose symptoms from other respiratory conditions, such as sleep apnea, by evaluating the rate of oxygen decline and heart rate variability and automatically adjusting the sensitivity of the emergency alert system based on the user's past responses and medical history.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the wearable health monitoring device of the present invention in accordance with the disclosed architecture;

FIG. 2 illustrates a rear view of the lifesaving wearable device in accordance with one embodiment of the present invention;

FIG. 3 illustrates a close view of the miniature naloxone injector in a deployed position for delivering naloxone or any other similar drug in accordance with the disclosed structure;

FIG. 4 illustrates a flow chart depicting a process of injecting opioid antagonist drug in accordance with one embodiment of the present invention;

FIG. 5 illustrates a confirmation message displayed by the lifesaving wearable device for input by the user in accordance with the disclosed structure;

FIG. 6 illustrates a block diagram view showing the components used for providing power to the wearable device for operation in accordance with one embodiment of the present invention; and

FIG. 7 illustrates a flow chart depicting a process of contacting emergency services in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a wearable health monitoring device that can autonomously detect opioid overdoses, respiratory failure, and other life-threatening emergencies in real-time. There is a long-felt need for a device that continuously tracks vital signs, such as oxygen levels, heart rate, blood pressure, and respiration patterns, and automatically alerts emergency services when an individual is unresponsive. Additionally, there is a long-felt need for a wearable intervention system that not only detects emergencies but also administers life-saving treatment. Furthermore, there is a long-felt need for a device that integrates wireless connectivity to transmit real-time health data and GPS location to emergency responders. More specifically, there exists a long-felt need for a smart wristband that provides both automated and user-confirmed emergency response mechanisms. Also, there is a long-felt need for an energy-efficient, portable, and comfortable device that can be worn daily without causing discomfort. Finally, there is a long-felt need for a comprehensive health monitoring and emergency intervention solution that can be used by opioid users, sleep apnea patients, and individuals at risk of respiratory distress.

The present invention, in one exemplary embodiment, is a method for detecting and responding to opioid overdoses using a wearable health monitoring device. The method includes the steps of continuously monitoring a user's oxygen saturation, heart rate, and respiration patterns using a plurality of sensors, detecting a critical drop in oxygen levels and an absence of normal respiratory activity for a predefined duration, generating an alert signal that requires the user to confirm consciousness via a touchscreen prompt or physical interaction with the device, determining that the user is unresponsive if no response is received within a predetermined time period, automatically activating a built-in injection system comprising a retractable micro-needle injector and a miniature motor, wherein the micro-needle injector delivers a dose of naloxone to counteract the effects of an opioid overdose, and transmitting an emergency notification to emergency contacts and medical personnel, including real-time GPS location and vital sign data.

Reference will now be made in detail to the present preferred embodiments of the invention, examples of which are illustrated in the accompanying drawings. Wherever possible, the same reference numerals are used in the drawings and the description to refer to the same or like parts.

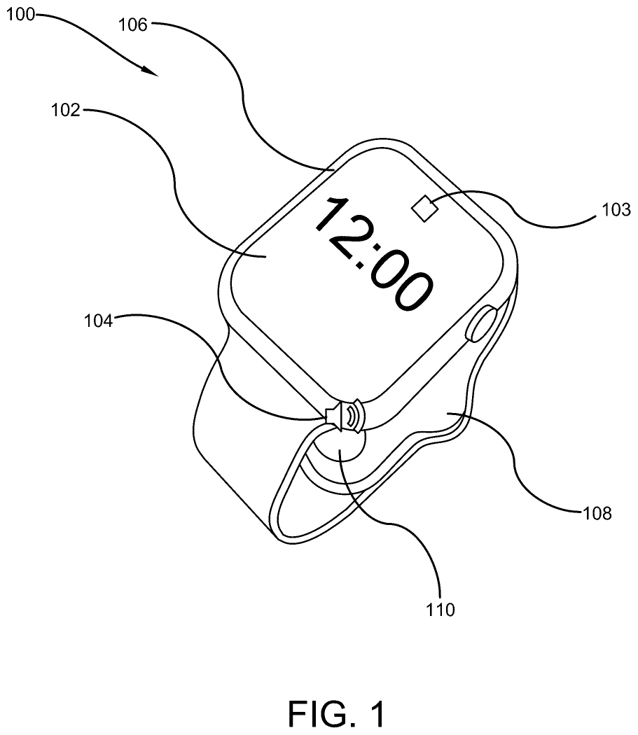

Referring initially to the drawings, FIG. 1 illustrates a perspective view of wearable health monitoring device 100 of the present invention in accordance with the disclosed architecture. The wearable health monitoring device 100 of the present invention is a wearable health monitoring device designed to detect and respond to life-threatening emergencies such as, but not limited to, opioid overdoses and respiratory failure. The health monitoring device 100 is configured to continuously track vital signs such as, but not limited to, oxygen levels, breathing, and blood pressure, and can automatically administer life-saving interventions when required.

More specifically, the wearable device 100 is adapted to be worn around the wrist of a user and has a touchscreen display 102. The display 102 displays various content 103 such as, but not limited to, vital signs, emergency alerts, and interactive prompts to allow users to interact with the device 100. A speaker 104 is disposed in the wearable device 100 and is designed to provide an audible alert to the wearer of the device 100. More specifically, the speaker 104 converts electrical signals from the device 100 into sound waves.

The display 102 may be any type of electronic touchscreen or non-touchscreen display. The display 102 may be a touch display for providing information and receiving input from the user. A water-resistant sealed housing 106 protects all the internal electronic components of the device 100 and enables users to wear the device 100 in water. The housing 106 is configured to be positioned against the user's body and a band 108 is configured to hold the housing 106 against the user's body or skin. The housing 106 is shaped and sized to fit on the desired target location for wearing the wearable band 100, such as on the wrist, ankle, neck, head, leg, or upper-arm of the user. The housing 106 is a protective case, shell, or platform to which the remaining parts are directly or indirectly attached, integrated, or housed. The band 108 may be any type of body-attachment band known in the art. In one embodiment, the band 108 may be secured, closed, bound, or joined joinable using a fastener 110. The fastener 110 may be comprised of a buckle, magnets, buttons, or other securing and locking mechanism. In one embodiment, the latch 110 is a clip or a clamp that allows the device 100 to be secured to a user's body or clothing.

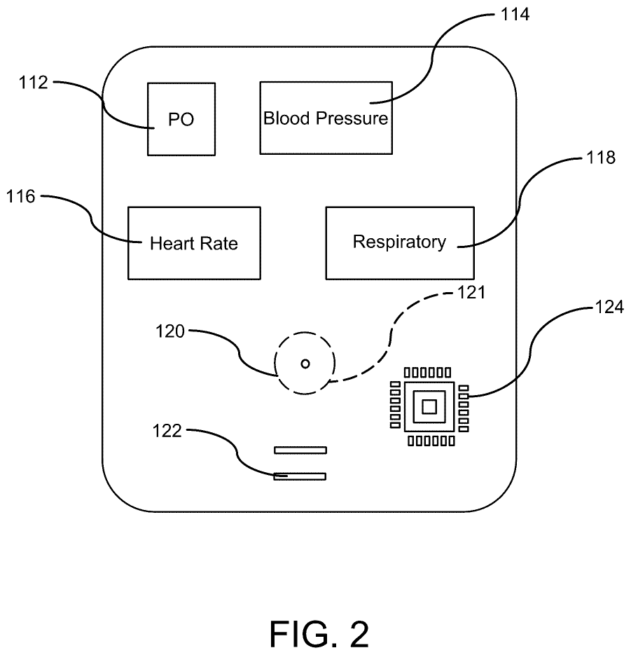

FIG. 2 illustrates a rear view of the lifesaving wearable device 100 in accordance with one embodiment of the present invention. The wearable device 100 includes at least one pulse oximeter sensor 112 to measure a user's oxygen saturation levels in the blood to detect hypoxia. The sensor 112 is preferably, but not limited to, an infrared LED photoplethysmography (PPG) sensor that measures how much light is absorbed by the user's blood to determine oxygen saturation levels.

The system is comprised of at least one blood pressure sensor 114, which may include an optical sensor, a pressure-based sensor, or a combination thereof, to measure the blood pressure of the wearer. The blood pressure sensor 114 may utilize photoplethysmography (PPG), tonometry, or oscillometric methods to determine blood pressure readings. A heart rate sensor 116 monitors heart rate and heart rhythm and may include an electrocardiogram (ECG) sensor, a PPG sensor, or a ballistocardiograph (BCG) sensor. A respiratory sensor 118 detects breathing patterns using bioimpedance, accelerometers, or a combination of both. The respiratory sensor 118 may include inductive plethysmography sensors, strain gauge sensors, or piezoelectric sensors to detect chest and abdominal movements associated with respiration. Additionally, the respiratory sensor 118 determines heart rate variability (HRV) to estimate respiration rate.

The device 100 may also be comprised of a naloxone injector 120 and is used as a built-in drug delivery system that administers a drug 121, such as but not limited to naloxone when opioid overdose symptoms are detected by the wearable device 100. An electric shock sensor 122 is configured to deliver a mild electric shock to stimulate the nervous system of the wearer of the device 100 if the wearer stops breathing. In different embodiments, the drug 121 may be nitroglycerin for heart attacks, lorazepam for seizure disorders, etc.

A wireless module 124 enables the wearable device 100 to communicate with smartphones and other similar devices. The wireless module 124 allows the device 100 to contact emergency services and provides the wearer's GPS location when the wearer is not breathing and not responding. It should be noted that different sensors can be in the form of electrodes which allow electricity to enter or leave the device 100. The sampling rate and size of the data sets detected and measured by different sensors may vary based on accuracy, resolution, and power utilization that is required by the wearable device 100.

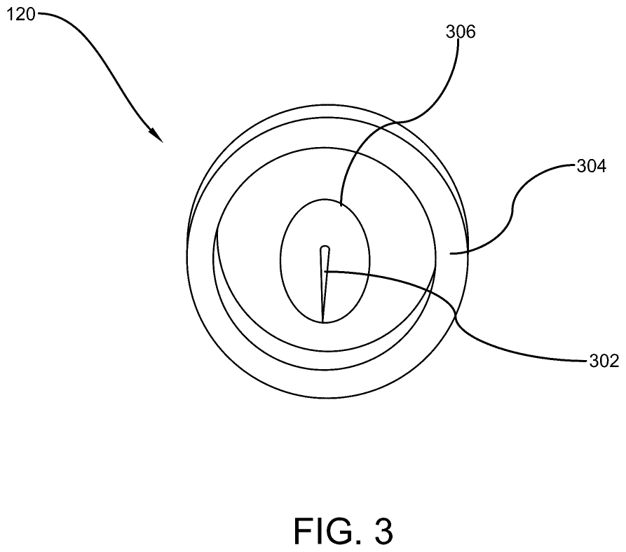

FIG. 3 illustrates a close view of the naloxone injector 120 in a deployed position for delivering naloxone or any other similar drug in accordance with the disclosed structure. As noted, the wearable device 100 has the built-in injection system 120 capable of delivering naloxone 121 (Narcan) if opioid overdose symptoms are detected. The naloxone injector 120 includes a retractable micro-needle injector 302 which can be extended to deliver naloxone subcutaneously or intramuscularly. A miniature motor 304 is automatically activated on activation of the built-in injection system 120 (when an opioid overdose is detected) to extend the micro-needle injector 302 to push the naloxone 121 into the user's body. The built-in injection system 120 may include a repository 306 for storing one or more doses of the opioid counter drug 121 and can be configured to provide one or more doses as required for the health of the wearer of the device 100. Preferably, the naloxone injector system 120 is configured to determine the appropriate amount of naloxone 121 to administer based on the wearer's physiological parameters and is capable of administering a second dose if the first dose does not restore normal breathing.

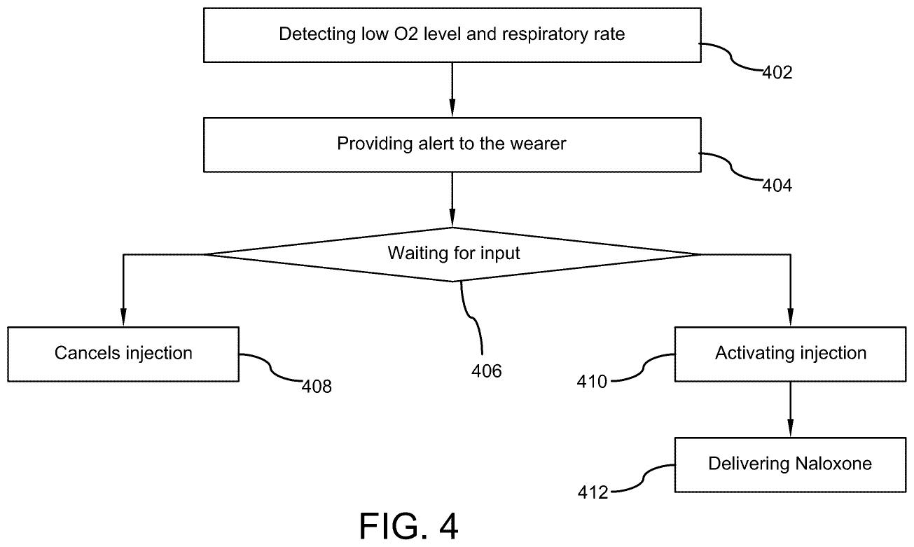

FIG. 4 illustrates a flow chart depicting a process of injecting an opioid antagonist drug in accordance with one embodiment of the present invention. Initially, various sensors of the wearable device 100 identify a critical drop in oxygen levels (such as below 90% level) and detect no or critically low respiratory activity for a set duration such as 20-30 seconds (Step 402). Upon completion of the set duration, the wearable device 100 generates a shock, sounds an alarm, and displays a warning asking if the wearer is okay (Step 404). It should be noted that the lifesaving device 100 includes an embedded algorithm that increases the sensitivity of the device to accurately detect opioid overdose and distinguish it from other conditions where breathing is decreased, such as sleep apnea. Preferably, the time duration in dropping the heart rate and the breathing is also used for determining opioid overdose.

Then, the wearable device waits a period of time, such as, but not limited to, 4-5 seconds, for the input (Step 406) and if the device 100 receives an input from the wearer, then the device 100 cancels the injection but continues monitoring of the wearer (Step 408). If no response is received from the user, then the wristband device 100 detects an emergency and automatically activates the built-in injection system 120 (Step 410). The needle injector 302 is extended and pressure is applied to the skin of the wearer to deliver the naloxone drug (Step 412).

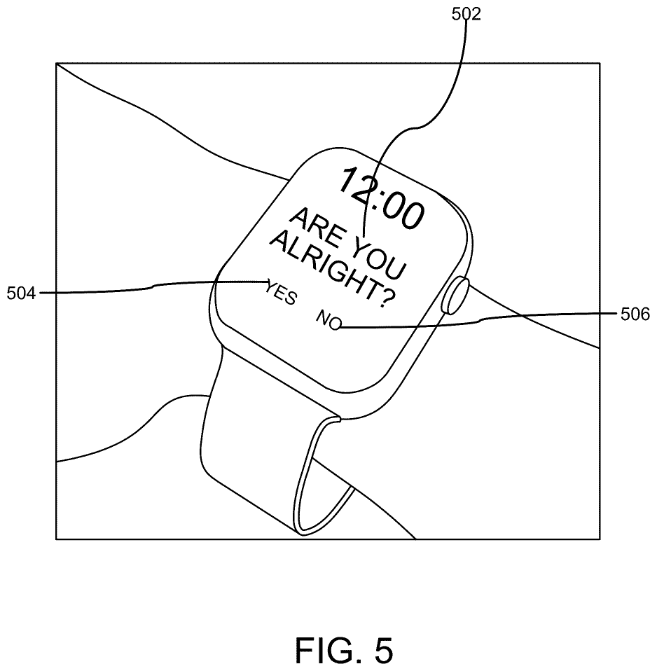

FIG. 5 illustrates a confirmation message displayed by the lifesaving wearable device for input by the user in accordance with the disclosed structure. As illustrated, the display 102 displays a question 502 such as, but not limited to, “Are you experiencing a medical emergency” requiring a response from the wearer to confirm consciousness. The question may be automatically displayed when abnormal vitals are detected by different sensors of the device 100. For confirmation, a “Yes” option 504 and a “No” option 506 are provided to the wearer and in case “Yes” option 504 is selected, then, no action is taken by the device 100, and vitals monitoring is continued. In case, “No” option 506 is selected by the wearer, then, a notification or call may be sent to one or more emergency services.

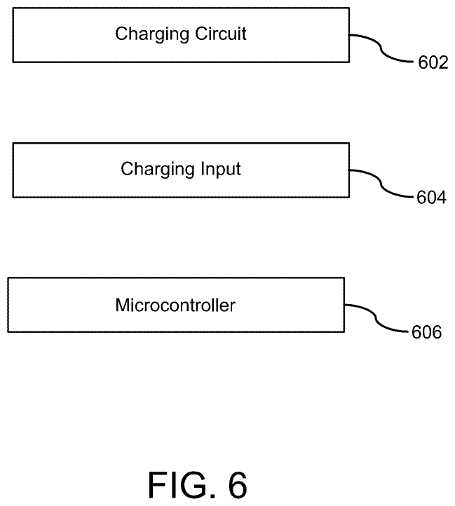

FIG. 6 illustrates a block diagram view showing the components used for providing power to the wearable device 100 for operation in accordance with one embodiment of the present invention. The device 100 includes a charging circuit 602 which may include wiring, hardware, and electronic logic and control systems to control charging of the device 100. The device 100 can be charged using a wired or wireless charging means to accommodate the requirements of different users. The charging circuit 602 may be connected to one or more charging inputs 604 such as the charging pins, that are configured to receive electric power. A microcontroller 606 manages the power consumption of the device 100 and prevents overheating and overcharging.

The microcontroller 606 is also configured to read signals from all the sensors in real-time and triggers alerts when one or more detected values are below or above the threshold range. The microcontroller 606 may use interfaces including, but not limited to, I²C (Inter-Integrated Circuit), SPI (Serial Peripheral Interface), and ADC (Analog-to-Digital Converter) for receiving signals from different sensors. The microcontroller 606 has baseline values that are used for comparison for generating alerts. As non-limiting examples, hypoxia is detected when the oxygen level drops below 90%. The microcontroller 606 may have adaptive duty cycles for different sensors 112, 114, 116, 118.

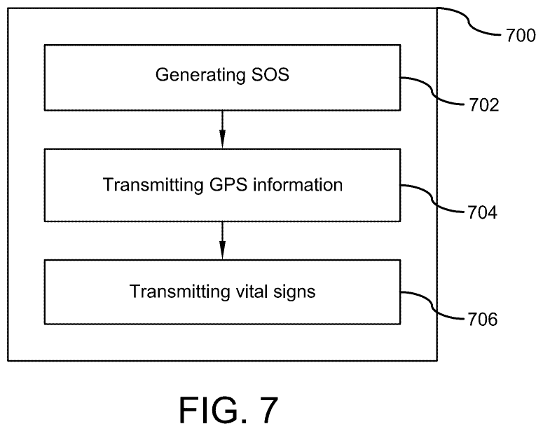

FIG. 7 illustrates a flow chart depicting a process 700 of contacting emergency services in accordance with one embodiment of the present invention. Initially, an SOS signal is generated when the wearer of the devices remains unresponsive for more than a predetermined time period (Step 702). The device 100 transmits the exact GPS coordinates to one or more emergency services and pre-set emergency contacts (Step 704). In case cellular connectivity is available, the device automatically dials 911 or a designated medical provider. Thereafter, the device transmits vital signs data to paramedics for quicker diagnosis upon arrival (Step 706).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “wearable health monitoring device”, “smart wristband for continuous vital sign monitoring and automatic opioid overdose reversal”, “lifesaving wearable device”, “wearable health monitoring device”, “health monitoring device”, and “device” are interchangeable and refer to the continuous vital sign monitoring and automatic opioid overdose reversal wearable device 100 of the present invention.

Notwithstanding the foregoing, the continuous vital sign monitoring and automatic opioid overdose reversal wearable device 100 of the present invention can be of any suitable configuration as is known in the art without affecting the overall concept of the invention, provided that it accomplishes the above-stated objectives. One of ordinary skill in the art will appreciate that the continuous vital sign monitoring and automatic opioid overdose reversal wearable device 100 as shown in the FIGS. are for illustrative purposes only, and that many other configurations of the continuous vital sign monitoring and automatic opioid overdose reversal wearable device 100 are well within the scope of the present disclosure. Although the dimensions of the continuous vital sign monitoring and automatic opioid overdose reversal wearable device 100 are important design parameters for user convenience, the continuous vital sign monitoring and automatic opioid overdose reversal wearable device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A wearable health monitoring device comprising:
a housing comprised of:
a display;
a pulse oximeter sensor configured to measure a user's oxygen saturation level;
a blood pressure sensor configured to measure a user's blood pressure;
a heart rate sensor configured to monitor a user's heart rate;
a respiratory sensor disposed configured to detect a user's breathing pattern;
a naloxone injector configured to administer a naloxone to the user upon detection of an opioid overdose symptom;
an electric shock sensor disposed configured to deliver an electric shock to the user upon detection of a respiratory failure; and
a wireless module;
a speaker that provides an audible alert; and
a band coupled to the housing.

2. The wearable health monitoring device of claim 1, wherein the pulse oximeter sensor comprises an infrared LED photoplethysmography sensor.

3. The wearable health monitoring device of claim 1, wherein the blood pressure sensor comprises an optical sensor or a pressure-based sensor.

4. The wearable health monitoring device of claim 1, wherein the heart rate sensor comprises an electrocardiogram sensor, a photoplethysmography sensor, or a ballistocardiograph sensor.

5. The wearable health monitoring device of claim 1, wherein the respiratory sensor comprises an inductive plethysmography sensor, a strain gauge sensor, or a piezoelectric sensor.

6. The wearable health monitoring device of claim 1, wherein the band is comprised of a fastener.

7. A wearable health monitoring device comprising:
a housing comprised of:
a display;
a pulse oximeter sensor configured to measure a user's oxygen saturation level;
a blood pressure sensor configured to measure a user's blood pressure;
a heart rate sensor configured to monitor a user's heart rate;
a respiratory sensor disposed configured to detect a user's breathing pattern;
a naloxone injector configured to administer a naloxone to the user upon detection of an opioid overdose symptom, the naloxone injector comprised of a retractable micro-needle injector and a motor;
an electric shock sensor disposed configured to deliver an electric shock to the user upon detection of a respiratory failure; and
a wireless module;
a speaker that provides an audible alert; and
a band coupled to the housing.

8. The wearable health monitoring device of claim 7, wherein the display is comprised of a touchscreen display.

9. The wearable health monitoring device of claim 7, wherein the housing is comprised of a water-resistant housing.

10. The wearable health monitoring device of claim 7, wherein the naloxone injector can administer a first dose and a second dose of the naloxone.

11. The wearable health monitoring device of claim 7, wherein the pulse oximeter sensor comprises an infrared LED photoplethysmography sensor.

12. The wearable health monitoring device of claim 7, wherein the blood pressure sensor comprises an optical sensor or a pressure-based sensor.

13. The wearable health monitoring device of claim 7, wherein the heart rate sensor comprises an electrocardiogram sensor, a photoplethysmography sensor, or a ballistocardiograph sensor.

14. The wearable health monitoring device of claim 7, wherein the respiratory sensor comprises an inductive plethysmography sensor, a strain gauge sensor, or a piezoelectric sensor.

15. A wearable health monitoring device comprising:
a housing comprised of:
a display;
a pulse oximeter sensor configured to measure a user's oxygen saturation level;
a blood pressure sensor configured to measure a user's blood pressure;
a heart rate sensor configured to monitor a user's heart rate;
a respiratory sensor disposed configured to detect a user's breathing pattern;
a naloxone injector configured to administer a naloxone stored in a repository of the naloxone injector to the user upon detection of an opioid overdose symptom, the naloxone injector comprised of a retractable micro-needle injector and a motor;
an electric shock sensor disposed configured to deliver an electric shock to the user upon detection of a respiratory failure; and
a wireless module;
a speaker that provides an audible alert; and
a band coupled to the housing.

16. The wearable health monitoring device of claim 15, wherein the motor extends and retracts the retractable micro-needle injector from the housing.

17. The wearable health monitoring device of claim 15, wherein the pulse oximeter sensor comprises an infrared LED photoplethysmography sensor.

18. The wearable health monitoring device of claim 15, wherein the blood pressure sensor comprises an optical sensor or a pressure-based sensor.

19. The wearable health monitoring device of claim 15, wherein the heart rate sensor comprises an electrocardiogram sensor, a photoplethysmography sensor, or a ballistocardiograph sensor.

20. The wearable health monitoring device of claim 15, wherein the respiratory sensor comprises an inductive plethysmography sensor, a strain gauge sensor, or a piezoelectric sensor.
